(19) United States
(12) Patent Application Publication
(10) Pub. No.: US 2019/0240637 A1
(43) Pub. Date: Aug. 8, 2019

(54) APPARATUS FOR COUPLING AND EMITTING LIGHT AND MATERIAL

(71) Applicants: Jong Duck PARK, Seongjeon-myeon (KR); Young Choon KIM, Gangjin-eup (KR)

(72) Inventors: Jong Duck PARK, Seongjeon-myeon (KR); Young Choon KIM, Gangjin-eup (KR)

(21) Appl. No.: 16/227,295

(22) Filed: Dec. 20, 2018

(30) Foreign Application Priority Data
Dec. 29, 2017 (KR) 10-2017-0184487

(51) Int. Cl. B01J 19/12; F41H 11/02; A01M 25/00; A01M 29/10; A01M 29/12; A01M 1/20; C01B 5/00; A61L 9/12

(52) U.S. Cl. CPC B01J 19/122; F41H 11/02; A01M 25/006; A01M 29/10; B01J 2219/1203; A01M 1/2022; C01B 5/00; A61L 9/12; B01J 2219/0875; A01M 29/12

(57) ABSTRACT

Disclosed is a light-material coupling and emitting apparatus for coupling and emitting light and a material. The present disclosure is to utilize light coupled with a gaseous material or a certain state of material. According to the present disclosure, light is coupled with a gas, a liquid, or any state (plasma state) of material, and the coupled light and the material are emitted toward a target object or a predetermined region. When reaching the target object or the predetermined region, the material reacts with another material existing around the target object or the predetermined region. According to one embodiment, when light coupled with a first material is emitted from the light-material coupling and emitting apparatus, the first material coupled with the light falls away from the light and then chemically reacts with a second material. The apparatus uses a specific effect caused by this chemical reaction.

### CROSS-REFERENCE TO RELATED APPLICATIONS

This present application claims priority to Korean Patent Application No. 10-2017-0184487, filed Dec. 29, 2017 the entire contents of which is incorporated herein for all purposes by this reference.

### FIELD

The present disclosure relates to an apparatus for coupling and emitting light and material. More particularly, the present disclosure relates to an apparatus for coupling a light ray and a material in any form, for example, a gaseous, liquid, or solid material, and emitting the coupled light and material.

### BACKGROUND

Unless otherwise stated herein, the contents set forth in this section are not prior art to the claims of this application. Thus, the contents set forth in this section should not be construed as being prior art just for the reason of being described in this section.

In physics, the term “light” refers to electromagnetic radiation of any wavelength, and light exhibits properties of waves, such as reflection, refraction, interference, diffraction, and Doppler effect. Light of relatively short wavelengths propagates in a straight line. When light propagates through one medium and encounters another medium, a part of the light is reflected and another part is refracted at the interface between different media. The controversy over whether light is a wave or a particle has been ongoing for a long time. The particle theory of light and the pulse theory of light were sharply opposed to each other in the 17th century, but the particle theory prevailed in support of Newton in the 18th century. In the 19th century, however, Thomas Young's “double-slit experiment” led to the wave theory, and Maxwell argued for the electromagnetic theory that light was a form of electromagnetic radiation. In the 20th century, the particle theory was again confirmed by the Planck's quantum theory, and afterwards the wave-particle duality of light, exhibiting properties of both waves and particles, is generally accepted. Light basically has properties of straight propagation, reflection, and refraction. Recently, research has been conducted on experiments for stopping or confining light by using the nature of light.

### SUMMARY

The present disclosure is to utilize light coupled with a gaseous material or a certain state of material (powder of a solid). Light is coupled with a gas, a liquid, or any state of material (plasma), and the coupled particles of the light and the material are emitted toward a target object or a predetermined region. When the coupled light and material reach the target object or the predetermined region, the material reacts with another material existing around the target object or the predetermined region.

In addition, in one embodiment, when light coupled with a material is emitted from a light-material coupling and emitting apparatus toward a target object, the material coupled with the light falls away from the light due to the collision of the light with the target object, and the materials separated from the light chemically react with each other or react with a material existing in the air.

In addition, in order to enable the light which is coupled with the material and is retained in a coupling unit to propagate forward through the coupling unit, temperature, current, or the like of the coupling unit is controlled to increase the transmittance of the coupling unit.

A light-material coupling and emitting apparatus according to one embodiment controls emission of light coupled with a material, thereby controlling a temperature around a target object and purifying air around the target object.

In particular, the light-material coupling and emitting apparatus supplies energy to a stimulation medium such as a liquid, a gas, or a semiconductor material and directs the light to the stimulation medium so that the light is transformed to have a single wavelength and a property of straight traveling. When the mass of the material to be coupled with the light is greater than a predetermined value, the intensity and wavelength of the light are controlled to offset an excessive mass so that the light and the material can be easily coupled with each other. In addition, the light-material coupling and emitting apparatus supplies energy to a stimulation medium to generate a single wavelength light and resonates the single wavelength light with another light wave to increase the coupling force between the light and the material. On the other hand, a diaphragm is installed on one side of a light-retaining material to control the mass of a material (for example, explosives) to be coupled with light, thereby causing friction with the light and enhancing an electrostatic effect, resulting in an increase in the coupling force between the light and the material.

In addition, in the apparatus, the coupling surface between the light and the material, at which the light stays, forms a boundary point state between transmission and absorption of light. At the boundary point state, the light is not transmitted, absorbed, and reflected.

In addition, the light-material coupling and emitting apparatus operates such that light rays coupled with materials are emitted to cross each other at a position in a space or to collide with an object such as a building so that the materials coupled with the light rays may fall away from the light rays and then chemically react with materials dispersed in the air.

In addition, light rays respectively coupled with different materials are emitted to collide with each other at a position in a space so that the materials may fall away from the light rays and the separated materials may chemically react with each other.

In addition, the light-material coupling and emitting apparatus couples light rays with hydrogen and emits the light rays coupled with hydrogen to a position in a space so that the light rays can collide with each other and the hydrogen separated from the light rays can react with oxygen existing in the air to form water molecules (H₂O). The water molecules disperse in the air, thereby creating a screen effect. When a display light is emitted toward the water molecules, a display screen can be displayed in the air.

As described above, the light-material coupling and emitting apparatus couples light with a material and emits the light coupled with the material, thereby purifying the environment around a target, utilizing a phenomenon occurring when a second material collides with the material that is separated from the emitted light when the emitted light collides with a target, delivering both light and a scent to around a person, obtaining a temperature control effect by causing heat absorption or heat generation in the air, or displaying a display screen in the air.

The effects, features, and advantages of the present disclosure are not limited to the effects, features, and advantages described above, and other effects, features, and advantages that are not mentioned above can be understood from the following detailed description or the configurations cited in the accompanying claims.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a block diagram illustrating a data processing process performed in a light-material coupling and emitting apparatus that couples light particles and material particles and emits the coupled light and material particles, according to one embodiment of the present disclosure;

FIG. 2 is a view illustrating an application example of the light-material coupling and emitting apparatus according to one embodiment of the present disclosure;

FIGS. 3A through 3E are views illustrating operations of the light-material coupling and emitting apparatus according to one embodiment of the present invention;

FIGS. 4A through 4E are conceptual diagrams illustrating coupling and emission (irradiation) of light and material in a system according to one embodiment of the present disclosure;

FIGS. 5A through 5D are views illustrating embodiments of the present disclosure in which light particles coupled with material particles are emitted and then the material particles fall away from the light particles when the coupled light and material particles reach a target destination; and

FIGS. 6A through 6G are views illustrating application examples of the present disclosure.

### DETAILED DESCRIPTION

The advantages and features of the present invention and the manner of achieving them will become apparent with reference to embodiments described in detail below and the accompanying drawings. The present disclosure may, however, be embodied in many different forms and should not be construed as being limited to the embodiments set forth herein. Rather, these embodiments are provided so that the present disclosure will be thorough and complete and will fully convey the concept of the invention to those skilled in the art. Thus, the present invention will be defined only by the scope of the appended claims. Like numbers refer to like elements throughout the following description herein. Further, in describing embodiments of the present disclosure, well-known functions or constructions will not be described in detail since they may unnecessarily obscure the gist of the present disclosure. The following terms are defined in consideration of the functions in the embodiments of the present disclosure and thus may vary according to the intentions of users, operators, or the like. Therefore, the definition of each term should be interpreted based on the contents throughout this specification

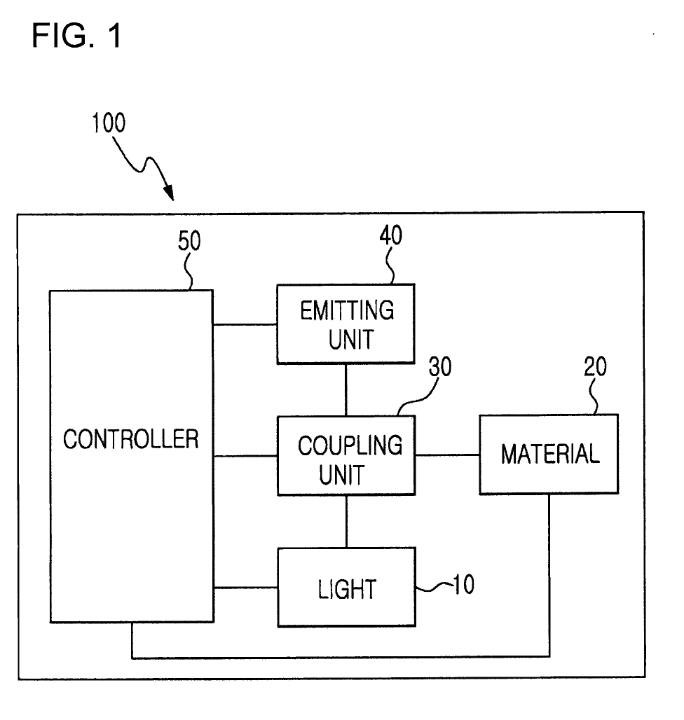

FIG. 1 is a block diagram illustrating a data processing process performed in a light-material emitting apparatus that couples light particles and material particles and emits coupled light and material particles, according to one embodiment of the present disclosure.

Referring to FIG. 1, a light-material coupling and emitting apparatus 100 according to an exemplary embodiment of the present invention includes a controller 50, an emitting unit 40, and a coupling unit 30. In an exemplary embodiment, light 10 to be coupled with a particular material is not limited to visible light but it may be radiation of any wavelength ranging from radio waves to gamma rays. In addition, in the exemplary embodiment, the apparatus 100 further includes a stimulated emission system for transforming spectral radiation to controlled radiation traveling in a straight line and having a narrow spectrum centered around a single wavelength. The stimulated emission system is controlled according to an exemplary embodiment. Alternatively, spectral radiation can be used as it is without involving a stimulated emission technique. The material 20 may be any form of material that can be coupled with light. For example, it may be a gas, a liquid, or a solid. The coupling unit 30 functions couple the light 10 and the material 20. The emitting unit 40 includes an X-axis driving unit and a Y-axis driving unit which are used to aim the coupled light and material at a certain target point. The controller 50 controls the emitting unit 30, the coupling unit 40, the light 10, and the material 20. To this end, the controller 50 controls a series of data processing operations necessary for coupling the light and the material.

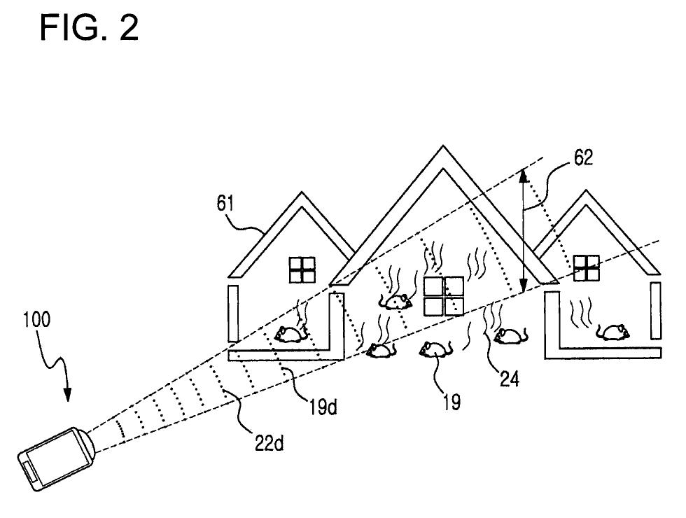

FIG. 2 is a view illustrating an application example of the light-material coupling and emitting apparatus according to one embodiment of the present disclosure.

As illustrated in FIG. 2, in order to eliminate mice from a building, an odorous material having a specific effect on mice is coupled with light, and the light and odorous material are emitted together to mice around the building. Then a beam of the coupled light and material hits the building and the material coupled with the light falls away from the light and disperses to repel or kill the mice.

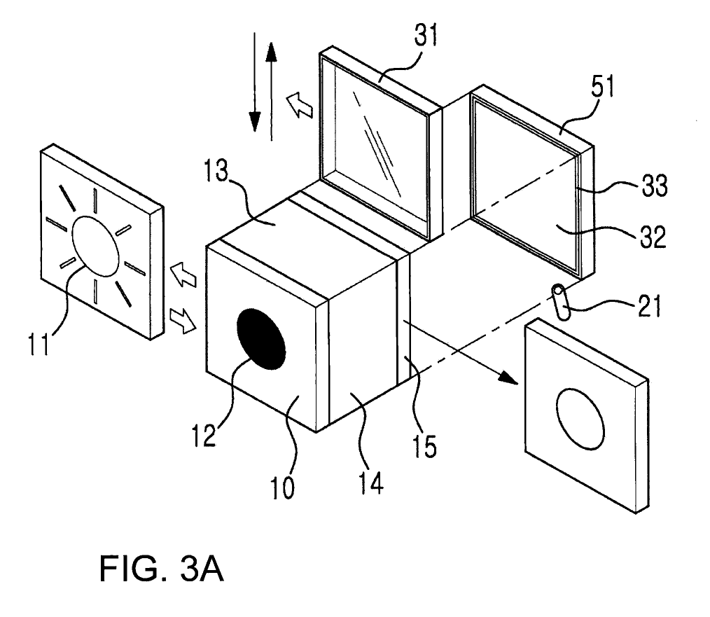

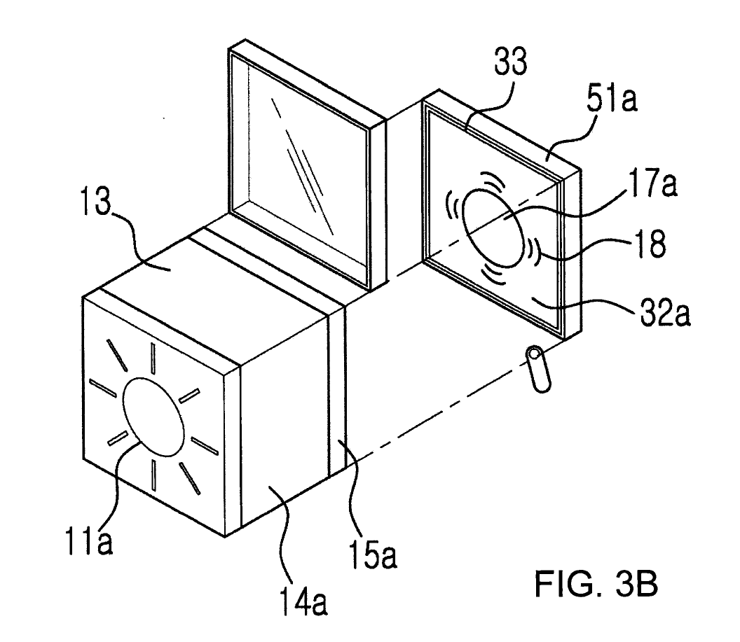

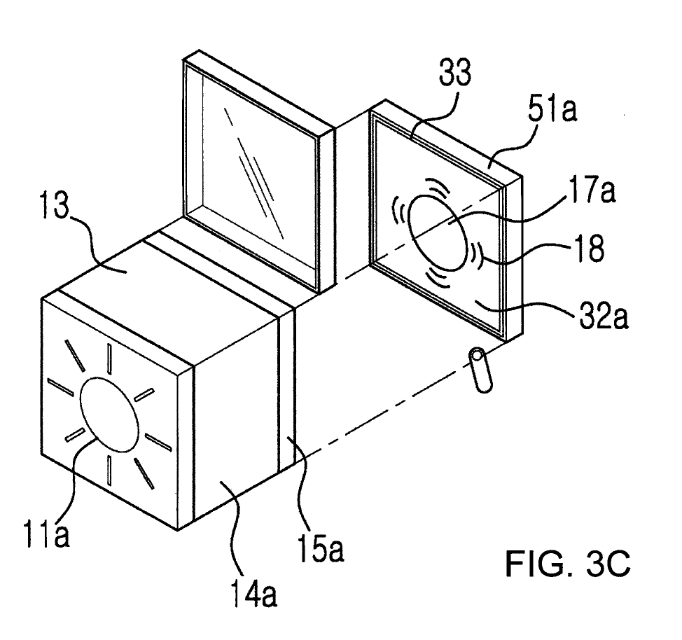

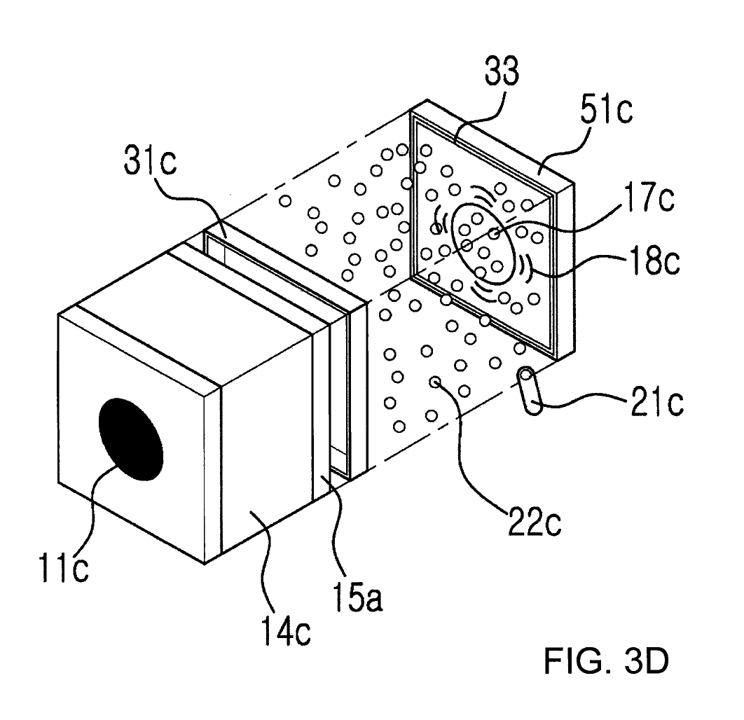

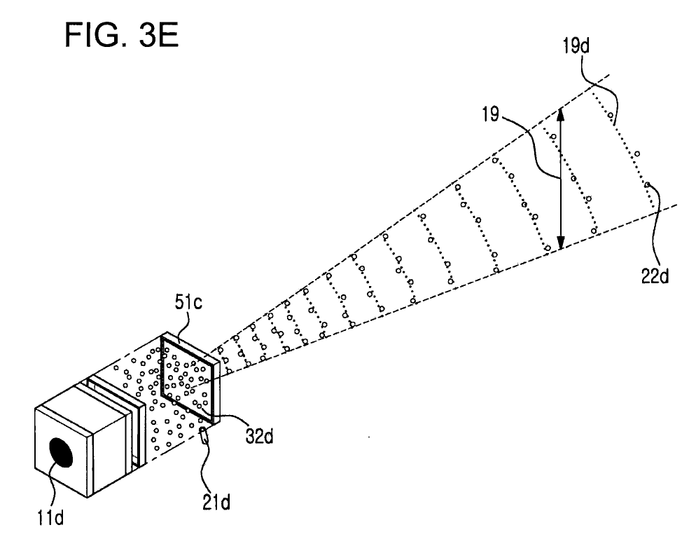

FIGS. 3A to 3E are views illustrating an operation process performed in the light-material coupling and emitting apparatus according to one embodiment of the present invention.

FIG. 3A illustrates components of the light-material coupling and emitting apparatus. Reference numeral 10 denotes light, reference numeral 11 denotes a light source in an ON state, and reference numeral 12 denotes light source in an OFF state. Reference numeral 13 denotes a stimulation medium to transform the light emitted from the light source 11 to a single wavelength ray traveling in a straight line. The stimulation medium 13 may be a liquid, gas, or semiconductor material, which may vary depending on embodiments. Reference numeral 14 denotes energy for causing stimulated emission. The energy 14 stimulates the stimulation medium to emit light of a single wavelength. In one embodiment in which the spectral radiation is used as it is, the energy 14 is not necessary. The controller 50 controls the energy supply state according to the kind and density of the stimulation medium and according to target values of various parameters for causing the stimulated emission. A reflector 15 is used to increase emission efficiency in a stimulated emission process. Reference numeral 31 denotes a confining unit for confining material particles and light particles. The confining unit 31 is moved in a predetermined direction when confining light particles and material particles and is moved in a different direction when allowing light to travel toward the coupling unit 32, thereby causing stimulated emission. The coupling unit 32 refers to a material to retain light therein. Reference numeral 21 denotes a feeding unit for feeding a material into the apparatus. The coupling unit 32 is tuned to have a boundary point among transmission, absorption, and reflection of light. That is, at the boundary point, neither transmission nor absorption of light does not occur. When light reaches the boundary point, the light does not propagate but stays and vibrates at a region due to properties of both particles and waves (electromagnetic waves) of light. That is, light cannot travel forward but stays therefor. This vibration leads to friction with light. Thus, in one embodiment, a diaphragm (vibration plate) 33 is further included to enhance a vibration effect at the boundary point in the material, thereby increasing the friction effect of the light.

Alternatively, the vibration effect is increased by a design in which the boundary point is disposed in a material that can vibrate at a frequency of tera hertz. When friction between light and a material occurs, an electrostatic phenomenon occurs, resulting in coupling between light particles and material molecules. When the mass of a material particle to be coupled with a first light ray is greater than a reference mass by a certain degree, the first light ray is resonated with a second light ray in intensity, amplitude, and wavelength to increase the coupling strength of light and material and offsetting the excessive mass of the material particle. On the other hand, when the vibration effect is adjusted to a certain target value, even material particles having a relatively large mass can be coupled with light particles. The controller 51 checks the resonant state of the wavelengths of a first light ray, a second light ray, . . . , and an n-th light ray, and controls energy supplied to the stimulation medium 14 to increase a resonating effect. When a control command for emitting a beam of light particles coupled with material particles is issued, the controller 51 controls temperature and current applied to the coupling unit to supply energy to the material so that the transmittance factor of the material is increased. As a result, the transmittance of the material increases to exceed the absorption rate and thus the light trapped in the material can be emitted.

In a system of FIG. 3B, when stimulated emission energy 14a is supplied to a stimulation medium and a light source 11a is turned on so that spectral radiation is emitted to a stimulated emission unit, the spectral radiation is transformed to controlled radiation centered around a single wavelength and having the property of traveling in a straight line due to the stimulated emission effect. In this case, according to one embodiment, the controller controls the stimulation medium, the stimulation energy, the light, and the like to meet target values of the wavelength and linearity of propagation of light. The light passing through the stimulated emission unit slows down and stays in a material (i.e., coupling unit 32a).

FIG. 3C illustrates a system in which the process performed in the system of FIG. 3B is repeated one more time. Through this repetition, it is possible to promote synthesis and resonance of light and enhance the coupling strength between the light and the material. The count of the repetitions may be increased from one to two, or to three, or to n times as necessary. On the other hand, through the coupling and resonance of the light, the amplitude of the light waves can be increased to a certain magnitude, so that a wind blowing effect can be obtained.

FIG. 3D illustrates a state in which a light source 11c is turned off. In this state, a single wavelength light having a property of traveling in a straight line, produced by the systems of FIGS. 13B and 13C, will stay in the material of the coupling unit 32c. Then, the confining unit 31 which is first positioned as in FIG. 13A is moved in one direction so as to be positioned like a confining unit 31c, so that the light and the material are confined in the same space. Reference numeral 21c denotes an inlet through which a material to be coupled with light is fed. According to one embodiment, a gas, a liquid, or solid (for example, red pepper powder) is fed into the apparatus through the inlet. Reference numerals 17c and 18c denote states in which the light stays in the coupling unit 32c. Due to the effects of the systems of FIG. 3B and 3C, i.e., due to the resonance of the intensity and wavelength of light, the coupling force between the light and the material and the intensity of the light are increased. Reference numeral 17c denotes a state in which particles and waves (electromagnetic waves) 18c of the light stay and vibrate at the surface of the coupling unit. Reference numeral 22c denotes a state in which the fed material is coupled with the light waves. The concept of coupling between light and a material will be described in more detail below. FIG. 3E is a diagram illustrating a process of emitting light coupled with a material. A controller 32d cancels a boundary point effect for confining light by adjusting temperature, current, energy, or other parameters, thereby allowing the light to be transmitted beyond the boundary point.

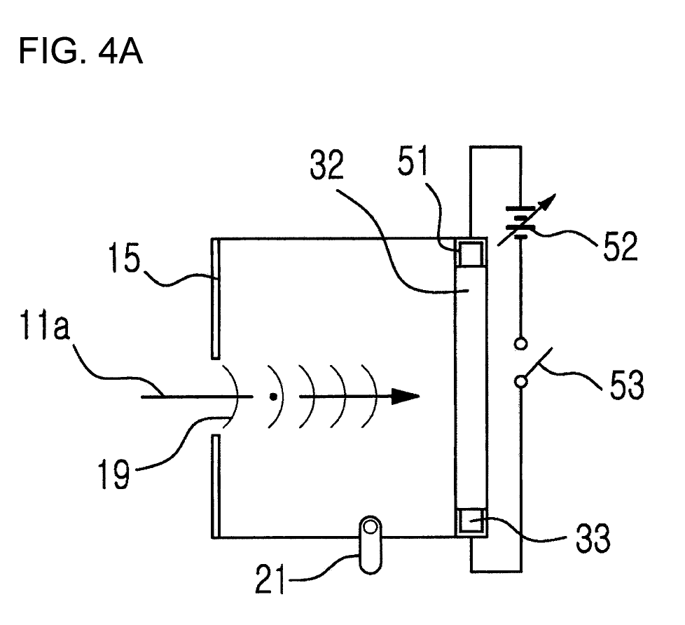

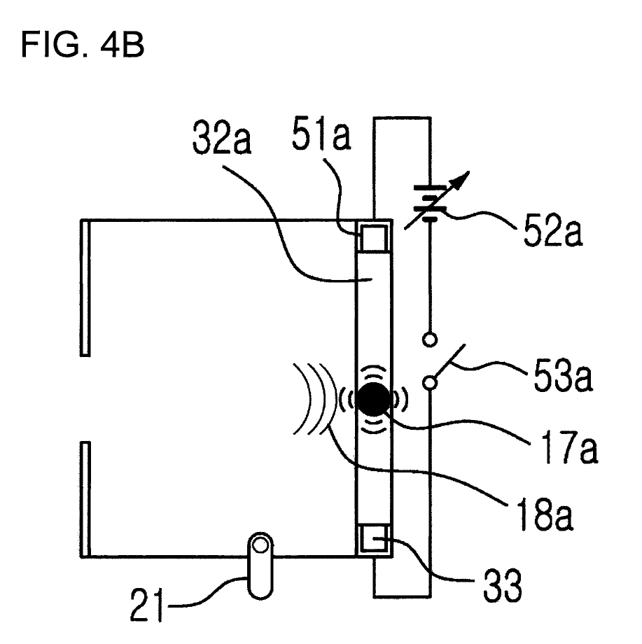

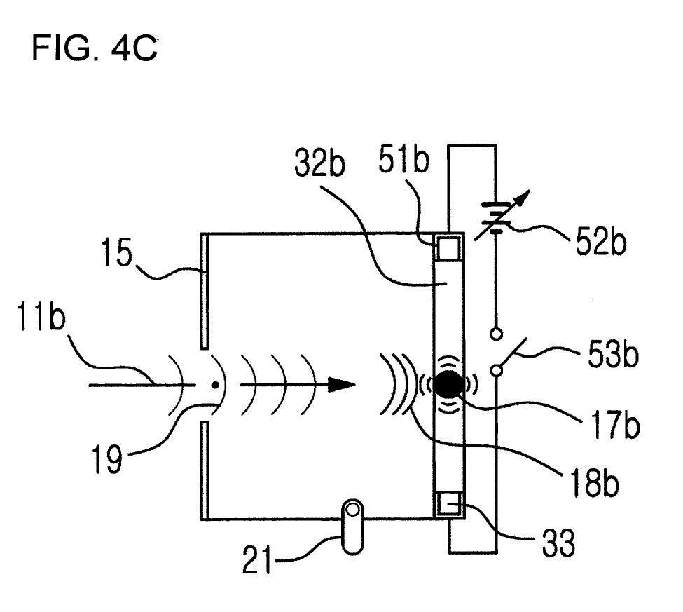

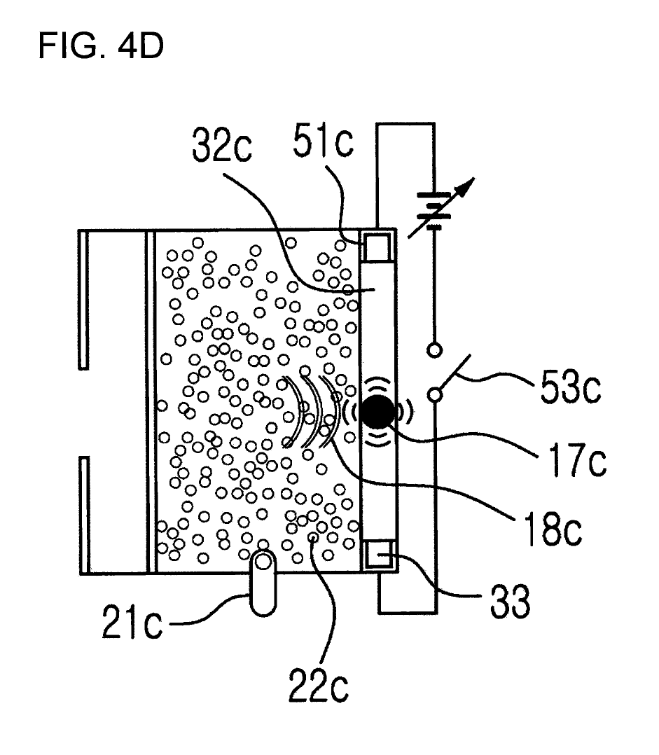

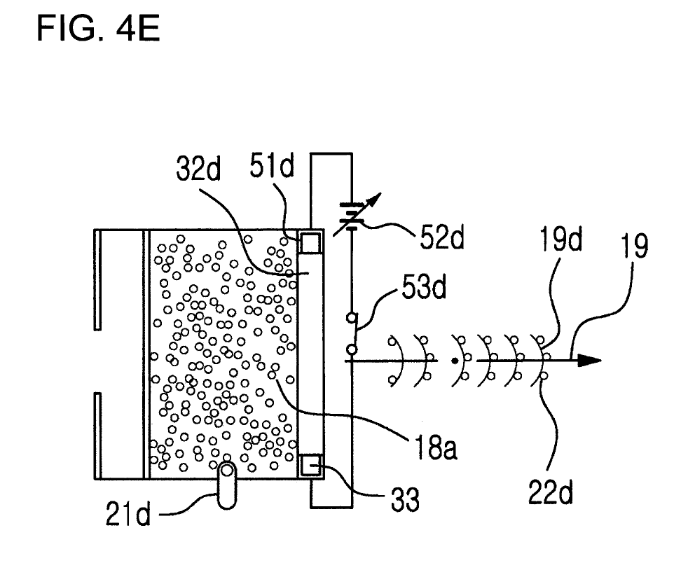

FIGS. 4A through 4E are conceptual diagrams illustrating the concept of coupling and emission (irradiation) of light and a material in a system according to one embodiment of the present disclosure.

To help with understanding of embodiments, the description of FIGS. 3A to 3E will be supplemented below. One of the conditions for coupling light and a material is that the light and the material must stay in the same space for a predetermined period of time. The speed at which light passes through a material is inversely proportional to the light absorption coefficient of the material. When light is incident on a certain material surface, it normally passes through or reflects from the material surface. However, when emission of the light is stopped, such phenomena (transmission and reflection) on the material surface immediately disappear. In embodiments, the expression “to confine the light” or “to retain the light” means a state in which light does not fade from the material surface but is trapped at the material surface when the emission of the light from a light source is stopped.

In FIGS. 4A through 4E, reference numerals 17a, 18a, 17b, 18b, 17c, and 18c do not denote afterimages of light but denote a light retention effect in each of the embodiments of the present disclosure. That is, the light retaining surfaces, i.e., the light and material coupling surfaces 32a, 32b, and 32c form a boundary point between transmission and absorption of light. Specifically, when reaching the boundary point between the transmission and the absorption, the light stays at the light retaining surface as if it were confined. Therefore, at this time, neither the transmission nor the absorption of light does not occur. At this time, even the reflection of light does not occur. The effect obtained by confining light will be described with reference to FIGS. 4A to 4E.

FIG. 4A illustrates a state in which a single-wavelength straight-line light 11 a is directed at the coupling surface 32. The coupling surface 32 is a material surface that is tuned to be at the boundary point at which none of absorption, transmission, and reflection of light do not occur.

FIG. 4B illustrates a state in which light 17a stays as if it were confined in a region 32a. At this time, light waves 18a also stay, and the light source 11a is turned off.

FIG. 4C illustrates a state in which light emitted from a light source 11b moves to points 17a and 18a near the region 32a. In the system of FIG. 4C, the light emitted from the light source 11 b is a single wavelength light traveling in a straight line.

FIG. 4D illustrates a state in which the light emitted from the light sources 11a and 11b combine with each other at the coupling unit 32c. At this time, since the wavelengths and the frequencies thereof are identical, the resonant effect of the light occurs. The vibration of the retained light is enhanced due to the resonant effect. At this time, an electrostatic phenomenon occurs due to the friction between the light and the coupling unit 32c.

Depending on the state of a material to be coupled with light, a diaphragm (vibration plate) 33 is controlled to enhance the electrostatic phenomenon. That is, the intensity of vibration of the material which is tuned to retain the light is enhanced to increase the friction between the diaphragm and the light. According to embodiments, the vibration frequency of the light and the vibration frequency of the material are controlled to be identical to cause resonance between the light and the material. In this way, the intensity of light and the coupling force between the light and the material are increased. The confining unit 31c is moved along a predetermined path toward one side so that a coupling space can be closed. A material to be coupled with light is fed into the space through an inlet 21c. In this case, the material to be coupled with the light may be a gas, a liquid, or a solid (in a specific state) according to objects of the embodiments. When negatively charged molecules (−) of the material approach the light, positive charges (+) of the light waves migrate to the material, and negative charges (−) migrate to the opposite side. Therefore, the negatively charged molecules (−) of the material and the positive charges (+) of the light waves couple with each other.

FIG. 4E illustrates a state in which a switch of a controller 53d is turned on to allow the light that is coupled with the material to propagate. As a result, the boundary point state between the transmission and the absorption of light abruptly changes to a transmission state in which the light can pass through the material. In order to emit the resonance-maximized light, the controller controls the instantaneous current supplied to the coupling unit 32d or increases the temperature of the coupling unit 32d. Then, the boundary point state in which the light is retained collapses, and the light can travel toward a target point. At this time, since the transmittance is maximized, the material 22d coupled with the light can be emitted along with light waves 19d.

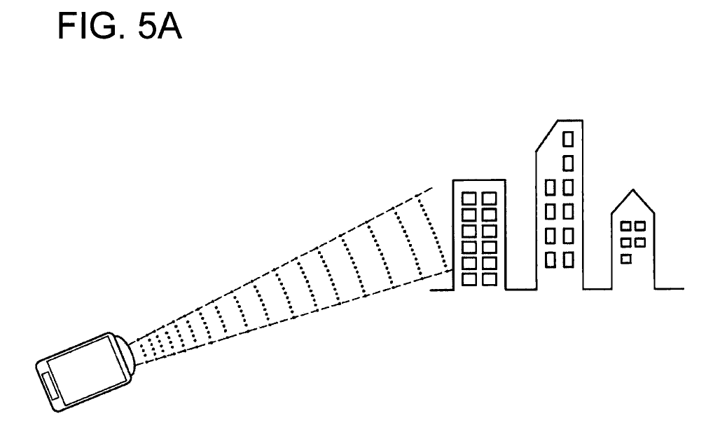

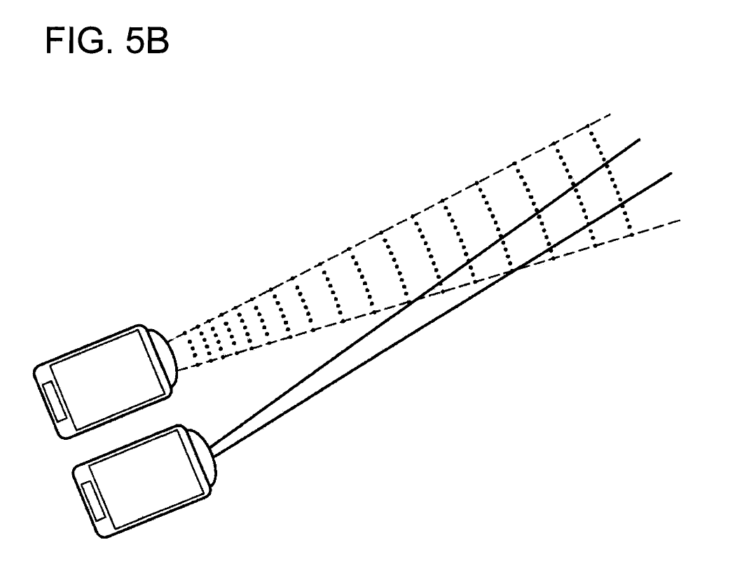

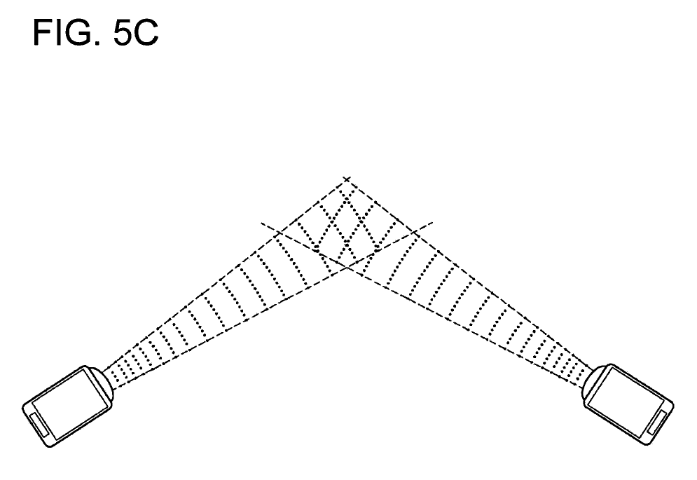

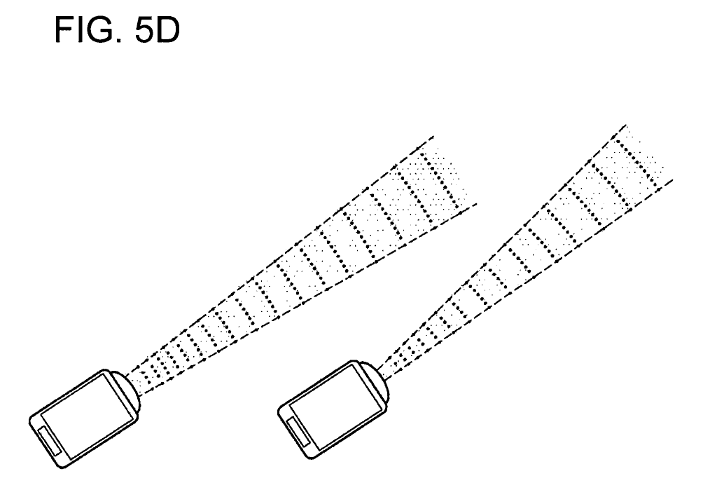

FIGS. 5A to 5D are views illustrating an operation example according to one embodiment of the present disclosure, in which light particles coupled with material particles are emitted and then the material particles fall away from the light particles when the coupled light and material coupled particles reach a target point.

A method of coupling a material and light, a light emitting unit, and a method of dissociating light and a material from each other are not limited to examples illustrated in FIGS. 5A through 5D. FIG. 5A illustrates an example in which light coupled with a material is directed at a building so as to collide with the building, and the material falls away from the light when the light collides with the building, so that the material disperses around the building.

FIG. 5B is an example in which a first light ray coupled with a material is emitted to a target point, a second light ray having a predetermined temperature is emitted to cross the first light ray, and the material falls away from the first light ray at an area where the first light ray and the second light ray cross each other. FIG. 5C is an example in which a first light ray coupled with an interest material and a second light ray coupled with the interest material are emitted to cross each other at a predetermined point in a space, the material falls away from the first light ray and the second light ray due to the collision between the first light ray and the second light ray, and the material falling away from the first ray and the second light ray chemically reacts with a certain material (for example, oxygen, nitrogen, etc.) dispersed in the air. FIG. 5D is an example in which visible light of a specific color is coupled with a material, and the coupled light and material are emitted together. There may be various combinations of emitted lights and materials and there may be various methods of separating the material from the coupled light.

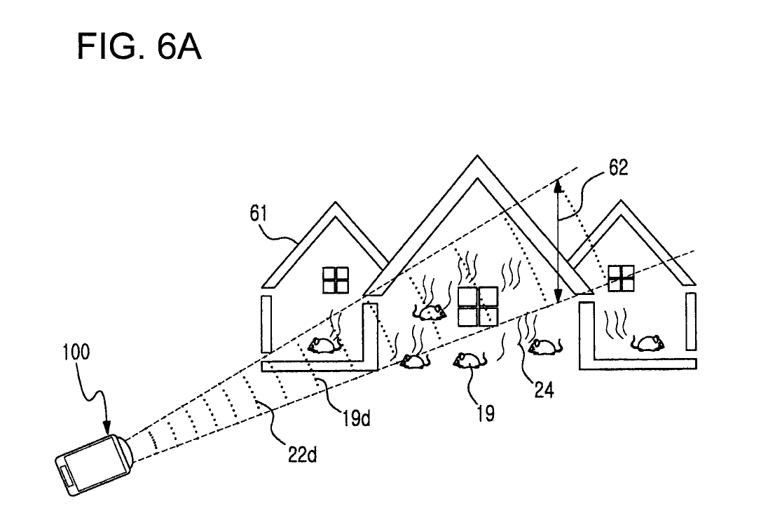

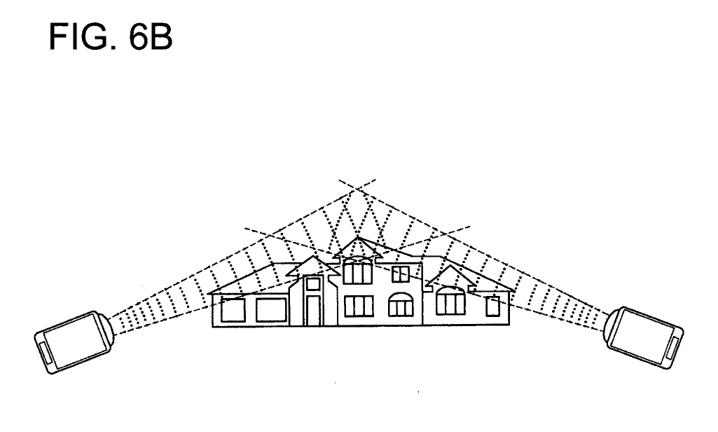

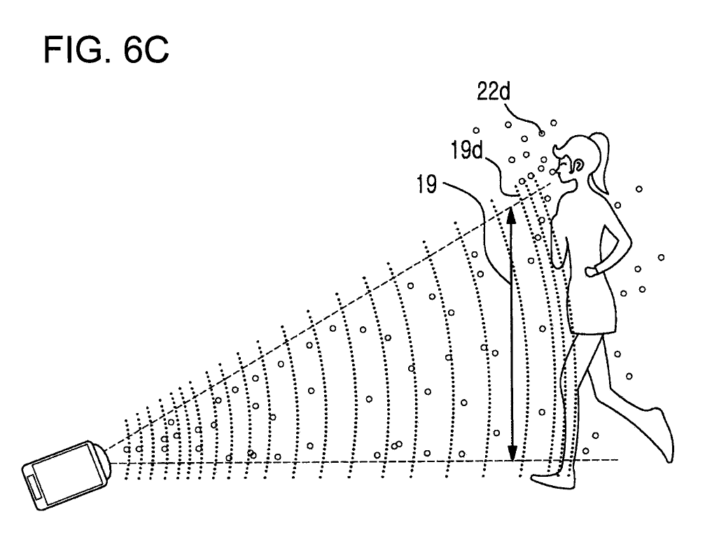

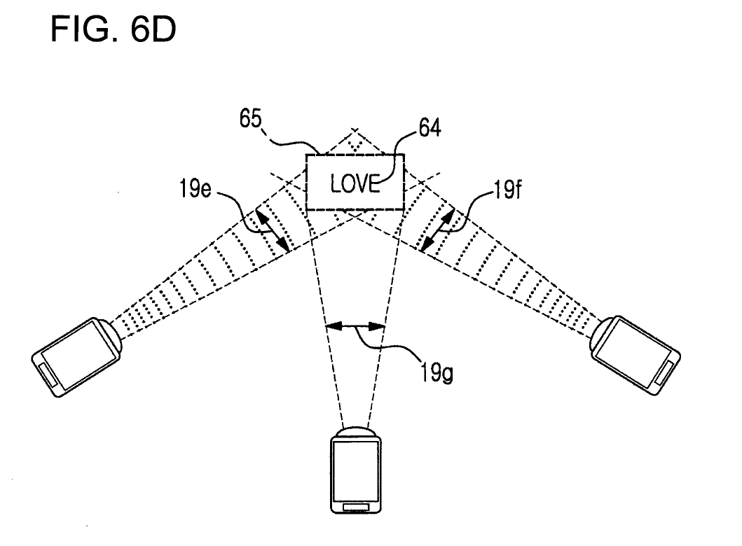

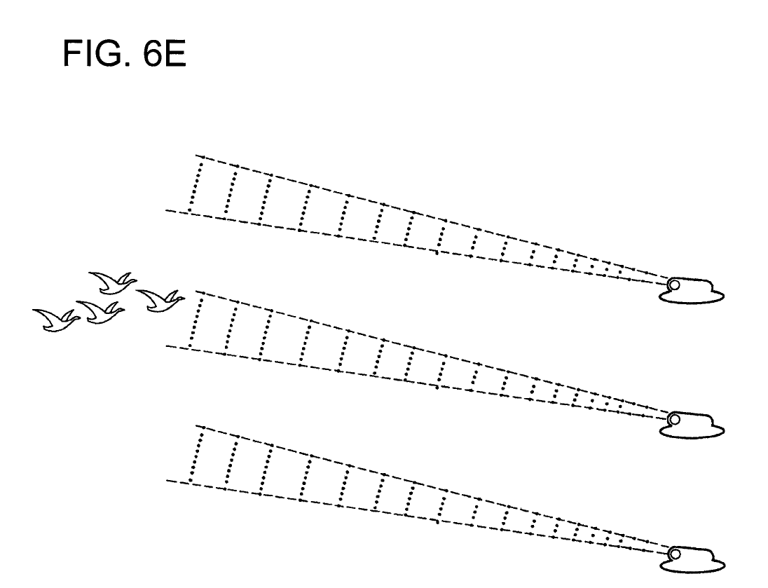

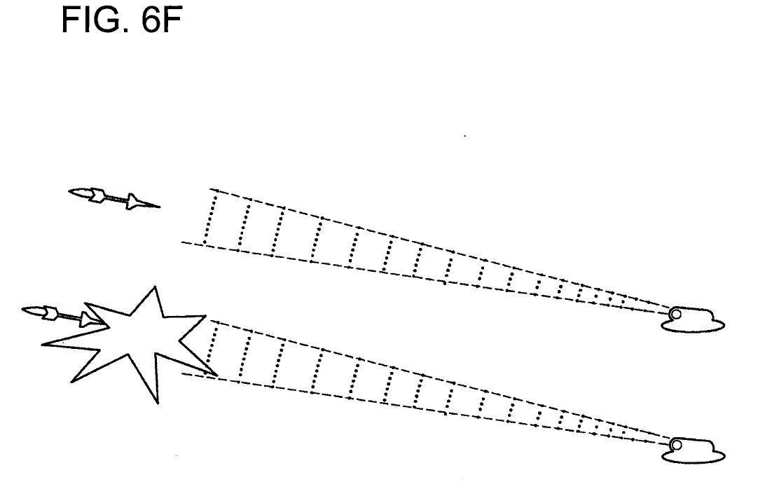

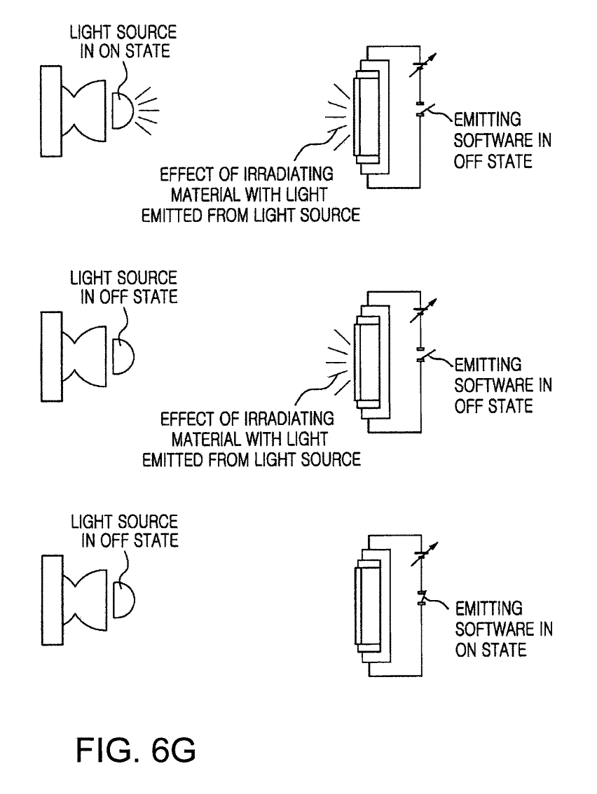

FIGS. 6A through 6G are diagrams illustrating application examples of the present disclosure.

FIG. 6A illustrates an example in which an odorous gaseous material 22 that mice or rats dislike or that has an insecticidal effect is coupled with a light ray, and the light ray is emitted to hit a building. When the light ray hits the building, the material falls away from the light ray and disperses around the building, thereby repelling or killing mice or rats. The application of the present disclosure is not limited to eradication of mice and rats. That is, the present disclosure can be used for elimination of mosquitoes, flies, and other insects.

FIG. 6B illustrates an example of adjusting the atmospheric temperature above a building by causing a chemical reaction of a material coupled with a light ray.

As illustrated in FIG. 6B, a first light ray emitted from the left side and a second light ray emitted from the right side cross each other at a point above a target building so that the first light ray and the second light ray can collide with each other. Thus, a material coupled with the first light ray and a material coupled with the second light ray chemically react with each other. This chemical reaction causes generation or absorption of heat, thereby obtaining an effect of controlling the temperature around the building. That is, by using a reaction heat of a chemical reaction, a cooling and heating effect is obtained. FIG. 6C illustrates an example of causing light rays to stay at a point on a coupling surface of a coupling unit and causing resonance between the waves of the light rays 19 and 19d, thereby increasing the amplitudes of the light waves to the extent that a person senses them. When the light rays are emitted to collide with a person, it is possible to obtain a wind blowing effect without natural wind.

On the other hand, according to one embodiment, a material to be coupled with a light ray is an odorous gas which emits a smell 22d of beach. In this case, when the light ray collides with a human body, the person can smell a beach.

FIG. 6D illustrates an effect of displaying a display screen in the air. As illustrated in FIG. 6D, according to one embodiment, light particles coupled with hydrogen are emitted from the left side and the right side to cross each other at a point in a space. Thus, the lights particles collide with each other at the point. At this time, the hydrogen falls away from the light particles and reacts with oxygen existing in the air, thereby forming water molecules (H₂O). The water molecules create a screen effect in the air. That is, when a display light is emitted from the center side toward the generated water molecules, a display screen can be formed in the air. In this case, when the display light for displaying the word “LOVE” is emitted, due to the properties of the light having a single wavelength and traveling in a straight line, which is produced by the system illustrated in FIG. 3, the effect of FIG. 6D can be obtained according to the distribution of the water molecules having a screen effect, the sharpness of the display light, and the intensity of the light. FIG. 6E illustrates an example in which the present disclosure is used to control the movement of migratory birds and the like in order to cope with avian influenza. As illustrated in FIG. 6E, a material that birds dislike is coupled with a light ray, and an emission angle of the light ray coupled with the material is adjusted by using a rotating unit so that the light ray can be emitted to an area in the air through which the birds move. When the birds enter the area, the light ray will hit or illuminate the birds, or the material coupled with the light ray influences the birds, thereby controlling a direction in which the birds fly.

FIG. 6F illustrates an example in which a light ray coupled with a material is emitted to a coordinated position in a radar machine by adjusting an emission angle of the light ray using an X-axis rotating unit and a Y-axis rotating unit so that the light ray hits a missile in a case where the missile is launched.

When particles of an explosive material or a bomb-like material are coupled with light particles and are then emitted toward a missile, it is possible to initiate explosion of the missile in the air by causing the collision between the missile and the material particles.

FIG. 6G illustrates an embodiment in which a light ray emitted from a lighting device such as an LED lamp is used as a light source. In this embodiment, an energy source for causing stimulated emission is turned off.

The present disclosure uses an effect of confining light on a material which is tuned to strongly keep light-material coupling such that light can be neither absorbed nor transmitted by the material. That is, a material on a surface of which transmission and absorption of light are in equilibrium is provided in a closed space of an apparatus. When the material in the apparatus is illuminated with light, the light stays at a point on the material surface and thus the material surface exhibits lighting effect. Further, if necessary, the equilibrium between the transmission and the absorption is broken so that the light can propagate through the material. At this time, the lighting effect of the material is canceled and the light is emitted from the material.

Although the present invention has been described with reference to exemplary embodiments, the exemplary embodiments are presented to describe the technical spirit of the present invention only for illustrative purposes and those skilled in the art will appreciate that various modifications and changes thereto are possible, without departing from the spirit of the present invention. Therefore, it should be understood that the protection scope of the present invention is defined by the accompanying claims rather than by the description which is presented above.

What is claimed is:

1. An apparatus for coupling and emitting light and a material, the apparatus comprising:
light to be coupled with a material;
a gaseous, liquid, or solid material to be coupled with the light;
a coupling unit configured to stop propagation of the light and to couple the light and the material;
an emitting unit configured to emit the light coupled with the material to a target; and
a controller configured to control the light, the material, the coupling unit, and the emitting unit.

2. The apparatus of claim 1, wherein the light is tuned to be transformed into a single wavelength light, according to a kind and a mass of the material to be coupled with the light, a distance to a target point, and a coordinate of the target point.

3. The apparatus of claim 1, wherein when the mass of the material to be coupled with the light is heavier than a predetermined mass, the light is synthesized with a second light so as to be resonated in intensity, vibration, and wavelength, thereby increasing a coupling force between the light and the material to offset an excessive mass.

4. The apparatus of claim 1, wherein a first material having a property of providing a state in which light is neither transmitted nor absorbed is provided on a side surface of a predefined space which the light enters, thereby stopping propagation of the light when the light reaches a surface of the first material so that the light stays on the surface of the first material, and then a second material to be coupled with the light is injected into the predefined space so that the light is coupled with the second material.

5. The apparatus of claim 1, wherein a vibration plate installed on one side of the material on which the light is retained is controlled to cause vibration of the material, so that molecules of the material are strongly coupled with particles of the light.

6. The apparatus of claim 1, wherein in a state in which the light is retained by a material having a boundary point where neither transmission nor absorption of light occurs, the material is heated to 70° C. or above to initiate the propagation of the light therethrough.

7. The apparatus of claim 1, wherein a first light ray is coupled with hydrogen and then emitted toward a predetermined target point in a space to collide with a second light ray or a certain object so that the coupling of the hydrogen and the first light ray is broken; the hydrogen decoupled from the first light ray chemically reacts with oxygen in the air to form water molecules (H₂O); the water molecules disperse to create a display effect in the air; and a display light is emitted from a center portion toward the water molecules so that a display screen is formed in the air.

8. The apparatus of claim 1, wherein particles of light are coupled with particles of an odorous gas or a pesticide and the coupled particles are emitted to a predetermined target in a predetermined distance so that the coupled particles collide with a certain object such as a building, a tree, an iron block, or a stone, thereby causing the particles of the odorous gas or the pesticide to be decoupled from the particles of the light so as to disperse in the air, thereby repelling rats, mosquitoes, flies, or pests.

9. The apparatus of claim 1, wherein an exothermic heat or an endothermic heat of a, reaction between a first material and a second material is calculated; a first light ray and a second light ray are coupled with the first material and the second material, respectively; and the first light ray and the second light ray respectively coupled with the first material and the second material are emitted toward a target position in a predetermined distance so that the first light ray coupled with the first material collides with the second light ray coupled with the second material or collides with a certain object to decouple the first material and the second material from the first light ray and the second light ray, thereby causing a chemical reaction between the first material and the second material to obtain an effect of adjusting a temperature of surrounding air through an exothermic reaction or an endothermic reaction.

10. The apparatus of claim 1, wherein the light is coupled with an explosive material formed to explode at a certain intensity of impact and the coupled light and material are emitted toward a target such as a missile to blow up the missile.

11. The apparatus of claim 1, wherein:
a second light is emitted to the light staying in the coupling unit so as to be resonated with the light, thereby controlling the wavelength of the light;
an aromatic material is coupled with the resonated light; and
the resonated light coupled with the aromatic material is emitted to a person so that the person can feel wind attributable to wavelengths of the light and smell an aromatic scent.

12. An apparatus for coupling and emitting light and a material, the apparatus being configured such that a material having a boundary point between transmission and absorption of light is installed on one side of a predetermined space, and light is emitted to the material from a light source, in which the emitted light stays in the material to give a lighting effect and the lighting effect is canceled by causing the light staying in the material to propagate through the material.
